(19) United States
(12) Patent Application Publication
(10) Pub. No.: US 2005/0209331 A1
(43) Pub. Date: Sep. 22, 2005

(54) Method of treatment of skin

(75) Inventor: Michael Kreindel, Haifa (IL)

Correspondence Address: BROWDY AND NEIMARK, P.L.L.C.;624 NINTH STREET, NW, SUITE 300, WASHINGTON, DC, 20001-5303, US

(73) Assignee: SYNERON MEDICAL Ltd., Yokneam Ellit (IL)

(21) Appl. No.: 10/944,420

(22) Filed: Sep. 20, 2004

Related U.S. Application Data
(63) Continuation-in-part of application No. 10/805,496, filed on Mar. 22, 2004.

(51) Int. Cl. A61K 31/195; A61K 7/135; A61N 1/30

(52) U.S. Cl. 514/561; 424/59; 604/20

(57) ABSTRACT

A method for treating a region of skin. An amount of at least one photolabile compound or a compound converted into a photolabile compound when present in the skin is applied to the region of skin. The photolabile compound generates a biologically active agent when in the skin and the skin exposed to sunlight. The applied compound is allowed to penetrate into the skin; and the region of skin is exposed to sunlight so as to generate the biologically active agent in the skin.

### CROSS-REFERENCE TO RELATED APPLICATIONS

This application is a continuation-in-part application of U.S. application Ser. No. 10/805,496, filed on Mar. 22, 2004, the entire contents of which are hereby incorporated by reference.

### FIELD OF THE INVENTION

This invention relates to methods for treating of skin.

### BACKGROUND OF THE INVENTION

5-Aminolevulinic acid (ALA), also called δ-aminolevulinic acid (δ-ALA), 5-amino-4-oxopentanoic acid, δ-amino-γ-keto-valeric acid, is a naturally occurring amino acid used as a photo-sensitizer in photodynamic therapy (PDT) of cancer and pre-cancer conditions such as actinic keratosis. ALA is applied to the skin surface to be treated in one or two applications of a 20% aqueous solution. After application of the ALA, the patient is advised to avoid exposure of the treated skin to sun light. 14 to 18 hours after application of the ALA, the patient returns to the care giver and the skin surface to be treated is exposed to blue light from a lamp for about 17 minutes.

In these treatments, a high concentration of ALA and high intensities of irradiation after application of ALA to the skin are applied. As a result, the subject often experiences pain, irritation, and a strong erythemic reaction.

U.S. Pat. No. 5,474,528 to Meserol describes PDT treatment in which a patch with a photo-sensitizer is applied to a dermal lesion and controlled irradiation is carried out using optical energy produced by a light source.

U.S. Pat. No. 5,441,531 to Azrate et al., discloses PDT treatment in which light in the range of 600-700 nm produced by a lamp is used.

U.S. Pat. No. 5,489,279 to Meserol describes a sealed applicator for applying a photo-sensitizer such as ALA to skin for PDT treatment.

U.S. Pat. No. 5,856,566 to Golub discloses use of colored ALA where the color is imparted by irradiation of ALA crystals.

U.S. Pat. No. 5,707,401 to Talmore describes a device for simultaneous PDT treatment and hyperthermia.

U.S. Pat. No. 5,776,175 to Eckhouse et al., describes use of pulsed incoherent light for PDT treatment of tumors.

U.S. Pat. Nos. 5,422,093, 5,234,940, 5,079,262, and 5,955,490 to Kennedy et al., describe the treatment of rapidly growing skin cells by application of ALA onto skin lesions and exposing the lesions to light.

### DESCRIPTION OF THE INVENTION

The present is based on the unexpected finding that certain treatments of skin may be carried out using patients in need of photodynamic therapy. In accordance with the invention, a photolabile compound such as ALA, or a compound that generates a photolabile compound when present in the skin, is applied to a skin region to be treated and is allowed to penetrate in the skin. The photolabile compound is a compound that generates a biologically active agent when present in the skin upon exposure to sunlight. After the applied compound has penetrated into the skin, the skin region is exposed to sunlight, so as to generate the biologically active agent in the skin without the undesired side effects associated with exposure to sunlight.

The present invention thus provides a method for treating skin. Conditions of the skin to be treated according to the invention include, without being limited thereto, acne, psoriasis, vascular and pigmented lesions, skin rejuvenation, actinic keratosis. According to a preferred embodiment, the treatment is a cosmetic treatment.

In accordance with the method of the invention, an amount of at least one photolabile compound of a compound converted into a photolabile compound when present in the skin is applied to a region of skin.

Interaction of the applied compound with sunlight generates a biologically active agent that stimulates skin metabolism and rejuvenation and kills bacteria on the skin surface, thereby preventing or at least reducing the level of acne. The cosmetic treatment may include, for example, skin bleaching, and pigmented lesion treatment.

The applied compound may be, for example, ALA, ALA derivatives and ALA precursors.

Non-limiting examples of ALA derivatives include lipophilic ester derivatives thereof, such as ALA hexyl ester, ALA benzyl ester, ALA pentyl ester or ALA methyl ester.

Other non-limiting examples of compounds that are converted into a photolabile compound in the skin include boronated protoporphyrin and derivatives of benzoprophyrine.

According to one embodiment, the method of the invention also includes applying to the region of the skin to be treated an amount of at least one filter substance. The term “filter substance” as used herein denotes any pharmaceutically acceptable substance that reduces either all intensity of sunlight or some of the wavelengths of the sunlight passing through it. Filter substances according to the invention include absorbing, reflecting and/or scattering substances, as known in the art. The amount of said filter substance applied to the skin should be effective to reduce the intensity of sunlight (or specific wavelengths of the sunlight) to which the skin is exposed.

As appreciated by those versed in the art, the type of filter substance to be selected depends on the region of the skin, e.g. the layer of the skin, and type of condition to be treated. For example, when the target region of the skin is the dermis (e.g. in case of skin rejuvenation treatment), a filter substance that filters out wavelengths shorter than 450 nm, having a penetration depth less than 0.1 mm, is preferably used. In another example, treatment of thick psoriatic plaques or acne requires a penetration depth of up to 0.5 mm. Thus, also in this case, a filter substance that filters out wavelengths shorter than 450 nm is used. In yet another example, for treatment of epidermal pigmented lesions a filter substance that filters out wavelengths around 400 nm is optimal.

The method of the invention may be used for cosmetic or therapeutic treatment of skin. Use of sunlight to irradiate the skin, as opposed to a specialized lamp, eliminates the need of the patient to return to the care giver after application of the agent for irradiation treatment. Moreover, by using sunlight, the treated skin may be exposed to the light over a period of several days or weeks, with each daily exposure being half an hour or more. Thus, by irradiating with sunlight, a significantly longer exposure time can be obtained than when the irradiation must be performed by a care giver. In some applications, such as cosmetic treatment of the skin, a longer exposure time may allow concentrations of the photosensitizing agent to be applied to the skin, lower than those typically used when treatment is combined with specialized lamps. As appreciated by those versed in the art, a lower concentration of the photosensitizing agent might reduce the side effects, e.g. pain and irritation as well as the erythemic reaction experienced by photodynamic treated patients.

In a preferred embodiment the agent is applied topically to the skin region. The agent may be formulated as oil, gel, ointment, paste, spray, sticks, cream or any other forms known in the art. To this end, the agent may be combined with thickening agents, gelling agents, suspension agents, emulsifiers, dispersing agents depending on the desired characteristics of the formulation. Those versed in the art of pharmacy will know how to select the specific excipients for proper formulation of the agent.

The agent may also be combined with other agents known to be used with topical formulations. For example, the agent may be combined with antioxidants such as beta-carotene, vitamin C, vitamin E, as well as with other skin care agents, e.g. glycolic acid or moister.

A preferred formulation according to the invention is a cream. The cream may include said at least one filter substance, or the filter substance may be applied to the region of the skin separately, also before exposure to sunlight.

According to one preferred embodiment the agent is applied to the region of the skin at a concentration lower than 20% w/w. According to a further preferred embodiment, the agent is applied to the skin at a concentration lower than 2% w/W.

The amount and schedule of treatment of the agent may vary and depends on considerations known to those skilled in the art.

According to one embodiment, for superficial effect, the agent may be applied to the skin region during morning hours, when there is mild irradiation by sunlight and in evening hours when deeper diffusion of the agent is required.

According to one embodiment, the treated skin region (e.g. the neck, face, hand etc.) is exposed to sunlight for at least half an hour a day, and preferably, for at least 2 hours a day.

According to yet another embodiment, the skin is exposed to the sunlight for at least 5 days, and preferably for at least 10 days.

The invention thus provides a method for treating a region of skin comprising:
(a) applying to the region of skin an amount of at least one photolabile compound or a compound converted into a photolabile compound when present in the skin, the photolabile compound generating a biologically active agent when the compound is in the skin and the skin is exposed to sunlight;
(b) allowing the applied compound to penetrate into the skin; and
(c) exposing said region of skin to sunlight so as to generate the biologically active agent in the skin.

1. A method for treating a region of skin comprising:
(a) applying to the region of skin an amount of at least one photolabile compound or a compound converted into a photolabile compound when present in the skin, the photolabile compound generating a biologically active agent when the compound is in the skin and the skin is exposed to sunlight;
(b) allowing the applied compound to penetrate into the skin; and
(c) exposing said region of skin to sunlight so as to generate the biologically active agent in the skin.

2. The method of claim 1, wherein said agent is 5-Aminolevulinic acid (ALA) or a derivative thereof.

3. The method of claim 2, further comprising applying to said region of skin at least one filter substance, prior to exposure of said region to sunlight.

4. The method of claim 3, wherein said filter substance is a light absorbing, light scattering, or light reflecting agent.

5. The method of claim 1, wherein the region is exposed to sunlight for at least half an hour on each of a number of days.

6. The method of claim 2, wherein the region is exposed to sunlight for at least half an hour on each of a number of days.

7. The method of claim 4, wherein the region is exposed to sunlight for at least half an hour on each of a number of days.

8. The method of claim 4, wherein the region is exposed to sunlight for at least two hours on each of a number of days.

9. The method of claim 5, wherein the region is exposed to sunlight for at least two hours on each of a number of days.

10. The method of claim 6, wherein the region is exposed to sunlight for at least two hours on each of a number of days.

11. The method of claim 4, wherein the number of days is at least 5.

12. The method of claim 5, wherein the number of days is at least 5.

13. The method of claim 6, wherein the number of days is at least 5.

14. The method of claim 10, wherein the number of days is at least 10.

15. The method of claim 11, wherein the number of days is at least 10.

16. The method of claim 12, wherein the number of days is at least 10.

17. The method of claim 1, wherein said photosensitizing agent is applied to the skin in the form of a cream.

18. The method of claim 2, wherein said ALA, a derivative of ALA or precursor thereof is applied to the skin in the form of a cream.

19. The method of claim 16, wherein said cream comprises at least one filter substance.

20. The method of claim 17, wherein said cream comprises at least one filter substance.

21. The method of claim 1 for use in cosmetic treatment of skin.

22. The method of claim 2, for use in cosmetic treatment of skin.

23. The method of claim 3, for use in cosmetic treatment of skin.

24. The method of claim 20, wherein the cosmetic treatment includes one or more of the cosmetic treatments selected from the group comprising skin rejuvenation, acne treatment, skin bleaching, and pigmented lesion treatment.

25. The method of claim 21, wherein the cosmetic treatment includes one or more of the cosmetic treatments selected from the group comprising skin rejuvenation, acne treatment, skin bleaching, and pigmented lesion treatment.

26. The method of claim 25, wherein the cosmetic treatment includes one or more of the cosmetic treatments selected from the group comprising skin rejuvenation, acne treatment, skin bleaching, and pigmented lesion treatment.
